(19) United States
(12) Patent Application Publication
(10) Pub. No.: US 2024/0377295 A1
(43) Pub. Date: Nov. 14, 2024

(54) SAMPLE PROCESSING CASSETTE

(71) Applicant: Esah Ali, Flushing, NY (US)

(72) Inventor: Esah Ali, Flushing, NY (US)

(21) Appl. No.: 18/492,428

(22) Filed: Oct. 23, 2023

Related U.S. Application Data
(60) Provisional application No. 63/501,672, filed on May 11, 2023.

(51) Int. Cl. G01N 1/31

(52) U.S. Cl. CPC G01N 1/312; B01L 3/502; B01L 2200/0605; B01L 2300/043; B01L 2300/0816

(57) ABSTRACT

Disclosed is a cassette designed for holding or processing samples effectively. The cassette includes a body for receiving the sample, and a lid adapted to securely cover the body. The body includes a body surface and a body side wall, a plurality of first body slots, and a plurality of second body slots. The lid includes a lid surface, a plurality of first lid slots, and a plurality of second lid slots. The plurality of first lid slots matches to the size of the first body slots, and a plurality of second lid slots matches to the size of the second body slots. The size of the first body slots can be different from the second body slots. The cassette is configured to have an optimum or increased flow of media and reagents to ensure thorough penetration and interaction with the sample.

### BACKGROUND

Sample processing cassettes are a widely used in laboratory settings for instance for diagnostic, drug screening, propagation and deep-dive histological, cellular and molecular analysis. Sample processing cassettes are employed to facilitate and possible sample operation, processing, analysis and storage.

Sample processing cassettes are generally made of plastic or other suitable materials such as high density acetyle polymer, comprise a chamber to house a sample, and optionally a lid or top to contain the sample. Often such cassettes also have areas to label and read the description of contents, such as patient name, type of sample, date collected or any other relevant information such as the type of sample, analysis or equipment needed or already employed in any sample processing. Labelling areas often present an advantage even the multi-chamber cassettes options. Same size gap sentilation mesh or same size slots may be featured on cassette sides as well as the bottom and hinged top for fluid exchange and draining.

Examples of sample processing cassette include for example tissues specimen processing cassette, in the context of histology and pathology. The histology and pathology type tissue specimen processing cassettes may comprise a small, rectangular, or square-shaped chamber. The cassette is used to hold and secure tissue samples, such as biopsies or surgical specimens, during processing and analysis in a laboratory. Various cassette designs are known in the art.

One consideration of relevance in selecting the right cassette type is the size and general nature or type of the sample which is to be processed. Attempting to take a one size fits all approach to cassette selection can lead to problems ranging from poor processing or loss of sample material falling out of the cassette to tissue damage and distortion by bars located within the chamber of the sample cassette as well as inadequate circulation or flow of growth media and other reagents.

Sample cassette design should facilitate proper immersion and exposure of samples to reagents and exchange of bi-products. The flow rate must be adjusted to ensure that reagents adequately penetrate the cassette's compartments and interact with the samples. Inadequate flow rates can lead to incomplete or inadequate processing, affecting tissue analysis and increase in cost due to need for repeat confirmatory experimentation. Poor cassette design can cause irregular flow rates leading to uneven processing, and inconsistent results. The circulation and flow rate should be adaptable to accommodate different cassette designs and sample size and types, ensuring that processing is effective regardless of variations in sample characteristics which would ultimately lead to cost reduction and time as well as improved quality and reliability of the processed sample generated data.

Despite these drawbacks, tissue cassettes remain an essential tool in sample processing. Therefore, there is need of a versatile cassette with improved overall performance and reliability while at the same time addressing at least some of the above identified problems of sample processing, downstream exploitation and reliability of the data.

### SUMMARY OF THE INVENTION

It is accordingly the object of the invention to provide a sample processing cassette configured for holding and processing both small and large samples. Moreover, it is also the object of the invention to provide a sample processing cassette configured for holding and processing both small and large samples of different types of samples.

Disclosed herein is a cassette comprising a body or chamber for receiving a sample and a lid adapted to securely cover the body. The body includes a body surface, a body side wall, a plurality of first body slots, and a plurality of second body slots.

The body side wall is extending in an upward direction from the body surface of the cassette. The plurality of first body slots and the plurality of second body slots are configured on the body surface of the body. The second body slots are of a different size from the first body slots.

The lid includes a lid surface, a lid side wall, a plurality of first lid slots, and a plurality of second lid slots. The lid side wall is extending in a downward direction from the lid surface of the cassette and detachably locks at the body side wall. The plurality of first lid slots and the plurality of second lid slots are configured on the lid surface of the lid. The first lid slots are of a same size as of the first body slots and the second lid slots are of a same size as of the second body slots. The second lid slots are of a different size from the first lid slots. each of the plurality of first body slots are substantially square in shape, and the second body slots are substantially rectangular in shape. In some embodiments, the walls of each of the plurality of first body slots are substantially conical and the walls of the second body slots are substantially conical.

Another aspect of the invention is to provide the first lid slots aligned in parallel to the first body slots, and the second lid slots are aligned in parallel to the second body slots to ensure proper flow of reagents in the cassette. Further, the second lid slots are configured to be surrounded by the first lid slots of the cassette. The second body slots are surrounded by the first body slots of the cassette.

Another aspect of the invention is the body of the cassette having a first body chamber, a second body chamber, a third body chamber, and a fourth body chamber. Each body chamber has plurality of second body slots. The second body slots configured in the first body chamber are in same direction as the second body slots configured in the third body chamber. The second body slots configured in the second body chamber are in same direction as the second body slots configured in the fourth body chamber.

In some embodiments of the invention there is provided a sample processing cassette with square shaped first body slots, and rectangular shaped second body slots.

In another aspect, there is provided a method of processing a sample in vitro, the method comprising the steps of:
a. obtaining a sample (102);
b. placing a sample (102) in a cassette (100) according to the present invention;
c. adding suitable for processing of sample reagents to the cassette under step b; and
d. processing the sample and reagent containing cassette under suitable conditions.

In another aspect, there is provided a method of diagnosing a disease or condition in vitro, the method comprising the steps of:
a. obtaining a sample (102);
b. placing a sample (102) in a cassette (100) according to the present invention;
c. adding suitable for processing of sample reagents to the cassette under step b;
d. processing the sample and reagent containing cassette under suitable conditions;
e. subjecting the incubated sample of step d. to further analysis such as micromanipulation, microtome manipulations, histology, histopathology, immunohistopathology, cytology, flow cytometry, cell sorting, microscopy, molecular testing, chromatography, microarray analysis, nucleotide sequencing, peptide sequencing, next generation sequencing or combinations thereof; and
f. diagnosing a disease or condition.

According to an embodiment of the present invention, the sample is one of an animal, plant, yeast, bacteria, insect, chemical or synthetic molecule. According to a further embodiment of the present invention, the sample is derived from an animal, plant, yeast, bacteria, insect, chemical, synthetic molecule or combinations thereof.

These and other features, aspects, and advantages of the present invention will become better understood with reference to the following description and appended claims.

### BRIEF DESCRIPTION OF DRAWINGS

Illustrative embodiments of the present invention are described herein with reference to the accompanying drawings, in which like numerals throughout the figures identify substantially similar components, in which:

FIG. 1 is an open top perspective view of a cassette, in accordance with embodiments of the invention; and

FIG. 2. is another open top perspective view of the cassette with samples, in accordance with an embodiment of the invention.

### DETAILED DESCRIPTION

For a further understanding of the nature and function of the embodiments, reference should be made to the following detailed description. Detailed descriptions of the embodiments are provided herein, as well as, the best mode of carrying out and employing the present invention. It will be readily appreciated that the embodiments are well adapted to carry out and obtain the ends and features mentioned as well as those inherent herein. It is to be understood, however, that the present invention may be embodied in various forms. Therefore, persons of ordinary skill in the art will realize that the following disclosure is illustrative only and not in any way limiting, as the specific details disclosed herein provide a basis for the claims and a representative basis for teaching to employ the present invention in virtually any appropriately detailed system, structure or manner. It should be understood that the devices, materials, methods, procedures, and techniques described herein are presently representative of various embodiments. Other embodiments of the disclosure will readily suggest themselves to such skilled persons having the benefit of this disclosure.

The present invention discloses a cassette for holding or processing a sample. Other features and advantages also are described as part of the invention. Accordingly, by employing the present invention, the cassette allows holding or processing of different samples with varied sizes, as further discussed herein.

As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein are solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers, or steps. Likewise, the term “comprising” is considered synonymous with the terms “including” or “containing” for applicable legal purposes. Any discussion of documents acts, materials, devices, articles, and the like are included in the specification solely for the purpose of providing a context for the present invention. It is not suggested or represented that any or all these matters form part of the prior art base or were common general knowledge in the field relevant to the present invention.

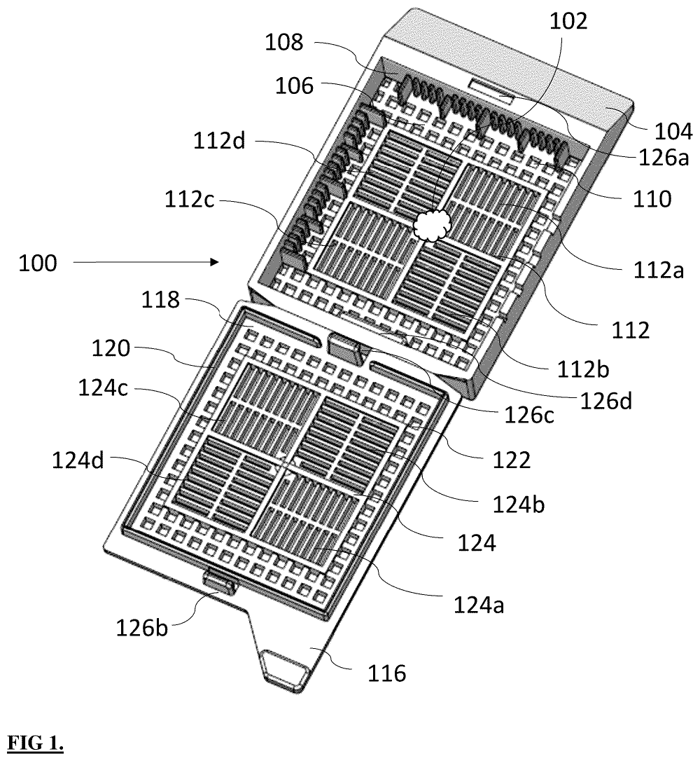

FIG. 1 is an open top perspective view of a cassette 100 for holding or processing a sample 102, in accordance with embodiments of the invention. The cassette 100 includes a body 104 for receiving the sample 102 and a lid 116 adapted to securely cover the body 104. The body 104 includes a body surface 106, a body side wall 108, a plurality of first body slots 110, and a plurality of second body slots 112.

The body side wall 108 is extending in an upward direction from the body surface 110 of the cassette 100. The plurality of first body slots 110 and the plurality of second body slots 112 are configured on the body surface 106 of the body 104. The second body slots 112 are of a different size from the first body slots 110.

In one embodiment, the plurality of first body slots 110 are square shaped and the plurality of second body slots 112 are rectangular shaped. In another embodiment, the plurality of first body slots 110 are rectangular shaped and the plurality of second body slots 112 are square shaped. However, it would be readily apparent to those skilled in the art that various shapes and sizes (such as rectangular, circular, oval, ellipsoidal, crescent, conical, triangular, square, pentagon, hexagon, rhomboidal, heptagon, octagon, trapezoid, kite and others) of the first body slots 110 and the second body slots 112 may be envisioned without deviating from the scope of the present application.

The lid 116 includes a lid surface 118, a lid side wall 120, a plurality of first lid slots 122, and a plurality of second lid slots 124. The lid side wall 120 is extending in a downward direction from the lid surface 118 and is configured to detachably lock at the body side wall 108. The plurality of first lid slots 122 and the plurality of second lid slots 124 are configured on the lid surface 118 of the lid 116. The first lid slots 122 are of a same size as of the first body slots 110. The second lid slots 124 are of a same size as of the second body slots 112.

In one embodiment, the plurality of first lid slots 122 are square shaped and the plurality of second lid slots 124 are rectangular shaped. In another embodiment, the plurality of first body slots 122 are rectangular shaped and the plurality of second lid slots 124 are square shaped. However, it would be readily apparent to those skilled in the art that various shapes and sizes (such as rectangular, circular, oval, ellipsoidal, crescent, conical, triangular, square, pentagon, hexagon, rhomboidal, heptagon, octagon, trapezoid, kite and others) of the first lid slots 122 and the second lid slots 124 may be envisioned without deviating from the scope of the present application.

In one embodiment, the plurality of first body slots 110, the plurality of second body slots 112, the plurality of first lid slots 122 and the plurality of second lid slots 124 are configured to provide optimized flow of the reagents. The plurality of first body slots 110, the plurality of second body slots 112, the plurality of first lid slots 122 and the plurality of second lid slots 124 are perforated/through-holes to allow passing of the reagents.

In one embodiment, the first lid slots 122 of the cassette 100 are aligned in parallel to the first body slots 110 to ensure optimize flow of reagents in the body 104. The second lid slots 124 are aligned in parallel to the second body slots 112 to ensure optimize flow of the reagents in the body 104. In one embodiment, the second lid slots 124 are surrounded by the first lid slots 122. The second body slots 112 are surrounded by the first body slots 110.

In one embodiment, the body 104 of the cassette 100 includes a first body chamber 112a, a second body chamber 112b, a third body chamber 112c, and a fourth body chamber 112d. Each body chamber has plurality of second body slots 112. The second body slots 112 configured in the first body chamber 112a are in same direction as the second body slots 112 configured in the third body chamber 112c. The second body slots 112 configured in the second body chamber 112b are in same direction as the second body slots 112 configured in the fourth body chamber 112d.

The first body chamber 112a is opposite to the second body chamber 112b, the second body chamber 112b is opposite to the third body chamber 112c, the third body chamber 112c is opposite to the fourth body chamber 112d and the fourth body chamber 112d is opposite to the first body chamber 112a.

In the similar manner, the lid 116 of the cassette 100 includes a first lid chamber 124a, a second lid chamber 124b, a third lid chamber 124c, and a fourth lid chamber 124d. Each lid chamber has plurality of second lid slots 124. The second lid slots 124 configured in the first lid chamber 124a are in same direction as the second lid slots 124 configured in the third lid chamber 124c. The second lid slots 124 configured in the second chamber 124b are in same direction as the second lid slots 124 configured in the fourth lid chamber 124d.

The first lid chamber 124a is opposite to the second lid chamber 124b, the second lid chamber 124b is opposite to the third lid chamber 124c, the third lid chamber 124c is opposite to the fourth lid chamber 124d, and the fourth lid chamber 124d is opposite to the first lid chamber 124a.

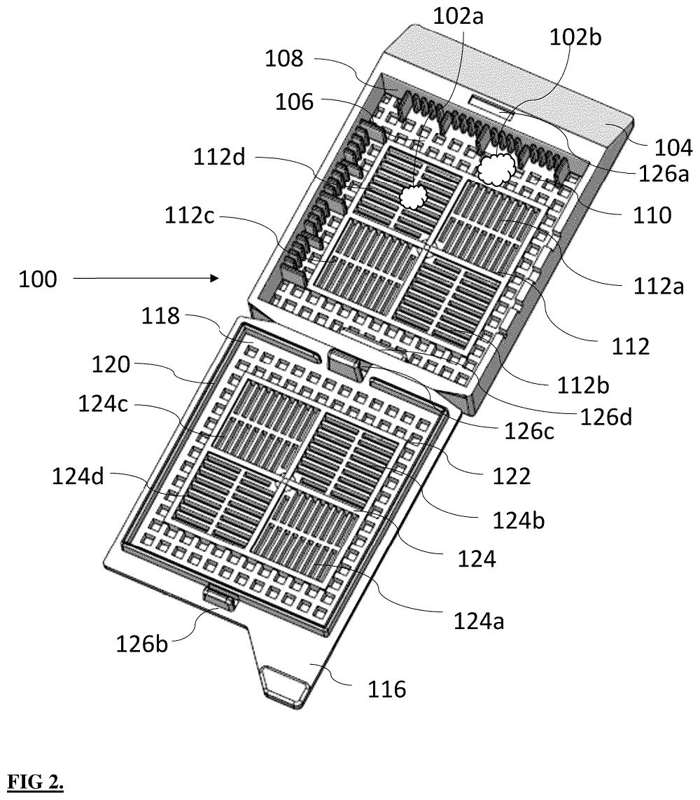

FIG. 2. is another open top perspective view of the cassette 100 with samples (102a, 102b), in accordance with an embodiment of the invention. The cassette 100 is used for holding or processing the samples 102a. 102b for histological analysis. Examples of the samples 102a, 102b included but not limited to animal, plant, yeast, bacteria, insect, plasma sample, blood sample, sputum sample, stool sample, lavage, synovial fluid, tissue sample, tumor biopsy sample, cell culture, chemical or synthetic molecule or combinations thereof.

The sample 102a is smaller in size than sample 102b. The cassette 100 allows reagents to pass through the slots and samples while maintaining the optimum flow of the reagents. The cassette 100 further includes attachment unit 126a, 126b, 126c, and 126d. The attachment unit 126a attaches to the attachment unit 126b and the attachment unit 126c attaches to the attachment unit 126d. Examples of the attachment unit 126a, 126b, 126c, and 126d includes but not limited to push locks, male-female locks, etc.

The present invention offers various advantages such as holding and processing of multiple tissue samples. Further, the cassette is designed to have an optimum flow of reagents to ensure thorough penetration and interaction with the sample.

The present inventors surprisingly observed that the use of a combination of different size or dimensions and types of slots in the sample processing cassette increased the flow rate of media and reagents within the chamber of the cassette. The present inventors also surprisingly observed that the use of a combination of different size or dimensions and types of slots in the sample processing cassette can be arranges in such a manner as to optimised the flow rate of media and reagents within the chamber of the cassette. The present inventors also surprisingly observed that the use of a combination of different size or dimensions and types of slots in the sample processing cassette can be arranges in such a manner as to achieve a synergistic effect in flow rates of media and reagents within the chamber of the cassette. The present inventors considered and demonstrated experimentally that it is advantageous to the incubated or screened samples to be housed within a chamber of a cassette incorporating an optimal flow of media and reagents for the particular sample which is successfully achieved by the combination of different size or dimensions and types of slots in the sample processing cassette of the present invention.

The different slots can be of any shape or dimension such as those selected from but not limited to the group comprising of rectangular, circular, oval, ellipsoidal, crescent, triangular, square, pentagon, hexagon, rhomboidal, heptagon, octagon, trapezoid, kite and others. In some embodiments the slots can be of any shape or dimension such as those selected from the group comprising rectangular, circular, oval, ellipsoidal, crescent, conical, triangular, square, pentagon, hexagon, rhomboidal, heptagon, octagon, trapezoid, kite and combinations thereof. The different slots of any shape or dimension can further comprise walls or sides which are substantially vertical. In some embodiment the slots are an open-ended cone also referred to herein as conical slots or open-ended conical slots. In some embodiment the slots are an open-ended cone also referred to herein as inverted conical slots or inverted open-ended conical slots. In some embodiments, the walls or sides of the slots are arranged at an angel. In some embodiments, one or more of the walls or sides of the slots are arranged at an angel.

The present inventors observed unexpectedly that improved circulation of fluid around a sample could be enhanced further by targeting various areas or parts of a sample which is deemed to be, in need, of greater access to fluid. The corollary was also unexpectedly observed as a benefit that is the slots can be arranged in a manner to enable reduced flow of fluid in areas or parts of the sample which would benefit from lower or reduced fluid flow. In some embodiments, the open-ended conical slots are arranged to act as funnels in attaining targeted directional transportation of fluid around the chamber. In some embodiments the open-ended conical slots are arranged to target specific areas of a sample wherever greater and/or reduced fluid flow is deemed beneficial to the target area.

As used herein the term “media and reagents” is intended to be interchangeable with the term “fluid” and means cell culture which is adapted for physiologically-relevant environments for diverse animal, plant, insect, yeast, bacteria cultures. The media is also intended to be suitable for 3D cell culture, cell growth maintenance, primary cell culture, stem cell culture, insect culture, phosphate buffered saline (PBS) and others. It is also intended that the fluid may also contain different reagents such as chemicals or synthetic molecules. In some embodiments of the present invention the sample processing cassette can be used for screening of candidate active ingredients. In some embodiments of the present invention the sample processing cassette can be used for high throughput screening of candidate active ingredients. In some embodiments of the present invention the sample processing cassette can be used to confirm or identify active ingredients. In some embodiments of the present invention the sample processing cassette can be determine the efficacy of the active ingredients. In some embodiments of the present invention the sample processing cassette can be used to determine the appropriate such as pharmaceutically efficacious dose of an active ingredients. There is an array of different candidate active ingredient and active ingredients which can be incorporated in the fluid and screened or tested using the sample processing cassette of the present invention and those would be familiar to the skilled in the technical art of drug discovery person.

In some embodiments of the present invention the sample processing cassette can be used to screen or test a plasma sample, blood sample, sputum sample, stool sample, lavage, synovial fluid, tissue sample, tumour biopsy, cytology specimen, cell culture, molecular testing or combinations thereof.

In one aspect of the present invention there is provided a method for processing a sample in vitro, the method comprising the steps of:
e. obtaining a sample (102);
f. placing a sample (102) in a cassette (100) as described herein;
g. adding suitable for processing of sample reagents to the cassette under step b; and
h. processing the sample and reagent containing cassette under suitable conditions.

In some embodiments, the processing of the sample is under a validated procedure. As used herein the term validated procedure may be determined by the relevant sample which is to be processed or for instance any health or market entry regulatory bodies such as FDA, EMA, local state and city Department of Health, Collage of American Pathologists and others.

In some embodiments, the processing of the sample is under a validated procedure for pathology sample processing. Example validated procedures for pathology sample processing would be familiar to the skilled person.

In one aspect, the present invention provides a sample processing cassette which can be used to diagnose a disease or condition in vitro by:
a. obtaining a sample (102);
b. placing a sample (102) in a cassette (100) as described herein;
c. adding suitable for processing of sample reagents to the cassette under step b;
d. processing the sample and reagent containing cassette under suitable conditions;
e. subjecting the incubated sample of step d. to further analysis such as micromanipulation, microtome manipulations, histology, histopathology, immunohistopathology, cytology, flow cytometry, cell sorting, microscopy, molecular testing, chromatography, microarray analysis, nucleotide sequencing, peptide sequencing, next generation sequencing or combinations thereof; and
f. diagnosing a disease or condition.

Without wishing to be bound by theory, it is proposed that the flow rate of fluid (also referred to as volumetric flow rate or media velocity) could be determined using the following mathematical formula:

$$Fluid flow rate ( Q ) = area of the hole / slot ( A ) \times velocity ( v )$$

Q=refers to the liquid flow rate measured as m³/s or L/s
A=refers to the area of the hole/slot (pipe or channel) in m²
v=refers to the velocity of the liquid in m/s

It is contemplated that the density of the media plays a role in determining the flow rate which could be determined using the following mathematical formula:

$$ρ = m / V$$

ρ=density of the fluid or media
m=mass flow rate
v=flow velocity of the mass elements

In one embodiment, where the slot is rectangular having dimensions: width of the slot is 1 mm and the height of the slot is 1 mm; the flow velocity is 100 m/s, and the liquid density is 1000 kg/m³—the volumetric flow rate of the media (m³) is determined as 0.36 m³/h.

In one further embodiment, where the slot is rectangular having dimensions: width of the slot is 5 mm and the heights is 0.5 mm, the flow velocity of 100 m/s; and the liquid density is 1000 kg/m³ the volumetric flow rate of the media (m³) is determined as 0.9 m³/h.

In some embodiments, the dimensions of each of the first body slot (110) and each of the first lid slot (122) being 1 mm×1 mm in opening, and wherein the space between each slot being in the range of from 0.5 mm to 1 mm.

In some embodiment, the dimensions of each of the second body slot (112) and each of the second lid slot (124) being in the range of from 0.5 to 5 mm in opening, and wherein the space between each slot being in the range of from 0.5 mm to 1 mm.

In some embodiment, each of the second body slot (112) and each of the second lid slot (124) are about 30% narrower and between about 100% to about 700% longer than each of the first body slot (110) and each of the first lid slot (122).

In some further embodiments, each of the second body slot (112) and each of the second lid slot (124) are about 40% narrower and between about 150% to about 600% longer than each of the first body slot (110) and each of the first lid slot (122).

In some further embodiments, each of the second body slot (112) and each of the second lid slot (124) are about 50% narrower and between about 200% to about 500% longer than each of the first body slot (110) and each of the first lid slot (122).

In some embodiments, the varied dimensions of slots (110) and (122) relative to slots (112) and (124) increase reagent flow in the body (104). In some embodiment, the wherein the varied dimensions of slots (110) and (122) relative to slots (112) and (124) optimize reagent flow in the body (104).

As used herein, the terms “body” and “chamber” are used interchangeably and mean a space where the sample is housed, contained or received, unless it is clear from the context or it is clearly stated that the term has a different meaning.

As used herein the term “sample” refer to a material from one of an animal, plant, yeast, insect, bacteria, chemical or synthetic molecule. In some embodiments, the sample is one of a plasma sample, blood sample, sputum sample, stool sample, lavage, synovial fluid, tissue sample, tumour biopsy sample, cell culture, cytology specimen, synthetic polymer, natural polymer, or combinations thereof.

In some embodiments, there is provided a sample processing cassette wherein the increase in fluid flow in the chamber is at least 1%, 1.5%, 2%, 2.5%, 3%, 3.5%, 4%, 4.5%, 5%, 10%, 15%, 20%, 25%, 30%, 35%, 40%, 45%, 50%, 55%, 60%, 65%, 70%, 75%, 80%, 85%, 90%, 95% or 100% or greater compared to standard tissue culture cassettes. Example standard sample processing cassettes include for example those described in US2018313811A1 or others.

In some embodiments, there is provided a sample processing cassette wherein the increase in fluid flow in the chamber is at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to standard tissue culture cassettes.

In some embodiments the second body slot (112) and second lid slot (124) lead to at least 2.5-fold increase in fluid flow compared to the first body slot (110) and the first lid slot (122).

In some embodiments of the present invention, the dimensions of each of the first body slot (110) and each of the first lid slot (122) being 1 mm×1 mm in opening, and wherein the space between each slot being in the range of from 0.5 mm to 1 mm.

In some embodiments of the present invention, the dimensions of each of the second body slot (112) and each of the second lid slot (124) being in the range of from 0.5 to 5 mm in opening, and wherein the space between each slot being in the range of from 0.5 mm to 1 mm.

In some embodiments of the present invention, each of the second body slot (112) and each of the second lid slot (124) are about 30% narrower and between about 100% to about 700% longer than each of the first body slot (110) and each of the first lid slot (122).

In some embodiments of the present invention, each of the second body slot (112) and each of the second lid slot (124) are about 40% narrower and between about 150% to about 600% longer than each of the first body slot (110) and each of the first lid slot (122).

In some embodiments of the present invention, each of the second body slot (112) and each of the second lid slot (124) are about 50% narrower and between about 200% to about 500% longer than each of the first body slot (110) and each of the first lid slot (122).

In some embodiments of the present invention, the varied dimensions of slots (110) and (122) relative to slots (112) and (124) optimise reagent flow in the body (104).

In some embodiments of the present invention, the varied dimensions of slots (110) and (122) relative to slots (112) and (124) increase reagent flow in the body (104).

In some embodiments, there is provided a sample processing cassette wherein the increase in fluid flow in the chamber is statistically significant compared to standard tissue culture cassettes.

As used herein, the term “significant” when referring to for example increasing, modifying, reducing, enhancing, remission, amelioration, prophylaxis, or reversal, that is statistically significant, not due to chance alone, which has a p-value of 0.05 or less. In particular, the term “significant” can have a p-value of less than 0.05, 0.04, 0.03, 0.01, 0.005, 0.001, etc., when referring to for example increasing the flow of fluid in the chamber, for example when compared with tissue culture cassettes on the market such as those described in US2018313811A1 (e. g. comparison with a “base line” level). Those of skill in the relevant art would be familiar with different statistical calculation approaches, examples include, t-test, z-test, sample test, O'Brien-Fleming method for normally distributed data etc.

As used in this application, the term “about” or “approximately” refers to a range of values within plus or minus 10% of the specified number. By way of non-limiting example, the term “about ten (10)” would encompass nine (9) to eleven (11) or 9-11.

The term “substantially” refers to up to 80% or more of an entirety, a value or percentage (%). Recitation of ranges of values herein are not intended to be limiting, referring instead individually to any and all values falling within the range, unless otherwise indicated, and each separate value within such a range is incorporated into the specification as if it were individually recited herein.

For the purposes of this disclosure, the term “aligned” means parallel, substantially parallel, or forming an angle of less than +45.0° degrees between for instance the different slots. For the purposes of this disclosure, the term “transverse” means perpendicular, substantially perpendicular, or forming an angle between 55.0 and 125.0° of the slots. Also, for the purposes of this disclosure, the term “length” means the longest dimension of a slot, cassette or lid. Also, for the purposes of this disclosure, the term “width” means the dimension of an object such as a lid or slots, from side to side. For the purposes of this disclosure, the term “above” generally means superjacent, substantially superjacent, or higher than another slot although not directly overlying the slots.

The use of any and all examples, or exemplary language (“e.g.,” “such as,” or the like) provided herein, is intended merely to better illuminate the embodiments and does not pose a limitation on the scope of the embodiments or the claims.

No language in the specification should be construed as indicating any unclaimed element as essential to the practice of the disclosed embodiments.

In the following description, it is understood that terms such as “first,” “second,” “top,” “bottom,” “up,” “down”, “upper”, “lower”, “above”, “below”, “beneath”, “front”, “back”, “over”, “under”, “left”, “right”, “end”, “upper lid”, “lower lid”, “edge” etc. are used with reference to the orientation of some of the components of the photovoltaic platform or photovoltaic system of the present invention. Since constituents or components in various embodiments described here can be positioned in a number of different orientations, locations of the floating surface, directional terminology is used for purposes of illustration only and is in no way limiting. The directional terminology is intended to be construed broadly, and therefore should not be interpreted to preclude components being oriented in different ways.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation.

The drawings and the forgoing description give examples of embodiments. Those skilled in the art will appreciate that one or more of the described elements may be very well combined into a single functional element. Alternatively, certain elements may be split into multiple functional elements. Elements from one embodiment may be added to another embodiment. For example, the orders of the processes described herein may be changed and are not limited to the manner described herein. Moreover, the actions of any process need not be implemented in the order shown; nor do all of the acts necessarily need to be performed.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art. Thus, the scope of the present disclosure is defined by the appended claims and includes both combinations and sub-combinations of the various features described hereinabove as well as variations and modifications thereof, which would occur to persons skilled in the art upon reading the foregoing description.

Further embodiments of the invention are encompassed in the following claims:

1. A cassette (100) for holding or processing a sample (102), the cassette comprising:
a body (104) for receiving a sample, the body comprising:
a body surface (106) and a body side wall (104) extending upward from the body surface;
a plurality of first body slots (110) configured on the body surface (106) of the body; and
a plurality of second body slots (112) configured on the body surface (106) of the body, wherein the second body slots (112) are of a different size from the first body slots (110);
a lid (116) adapted to securely cover the body, the lid comprising:
a lid surface (118) and a lid side wall (120) extending downward from the lid surface, further the lid side wall (120) detachably locks at the body side wall (108);
a plurality of first lid slots (122) configured on the lid surface of the lid, further the first lid slots (122) are of a same size as of the first body slots (110); and
a plurality of second lid slots (124) configured on the lid surface of the lid (116), further the second lid slots (124) are of a same size as of the second body slots (112).

2. A cassette according to claim 1, wherein the plurality of first body slots (110), the plurality of second body slots (112), the plurality of first lid slots (122) and the plurality of second lid slots (124) are configured to provide optimize flow of reagents.

3. A cassette according to anyone of claim 1 or claim 2, wherein the first lid slots (122) are aligned in parallel to the first body slots (3) to ensure optimize flow of reagents in the body (104).

4. A cassette according to anyone of claims 1 to 3, wherein the second lid slots (124) are aligned in parallel to the second body slots (112) to ensure optimize flow of reagents in the body (104).

5. A cassette according to anyone of the preceding claims, wherein the second lid slots (124) are surrounded by the first lid slots (122).

6. A cassette according to claim 5, wherein the second body slots (112) are surrounded by the first body slots (110).

7. A cassette according to claim 1, wherein the dimensions of the first body slots (110) are smaller relative to the dimensions of the second body slots (112).

8. A cassette according to claim 7, wherein the dimensions of the first lid slots (122) are smaller relative to the dimensions of the second lid slots (124).

9. A cassette according to anyone of claims 7 or 8, wherein the dimensions of each of the first body slot (110) and each of the first lid slot (122) being 1 mm×1 mm in opening, and wherein the space between each slot being in the range of from 0.5 mm to 1 mm.

10. A cassette according to anyone of claims 7 or 8, wherein the dimensions of each of the second body slot (112) and each of the second lid slot (124) being in the range of from 0.5 to 5 mm in opening, and wherein the space between each slot being in the range of from 0.5 mm to 1 mm.

11. A cassette according to anyone of claims 9 or 10, wherein each of the second body slot (112) and each of the second lid slot (124) are about 30% narrower and between about 100% to about 700% longer than each of the first body slot (110) and each of the first lid slot (122).

12. A cassette according to anyone of claims 9 or 10, wherein each of the second body slot (112) and each of the second lid slot (124) are about 40% narrower and between about 150% to about 600% longer than each of the first body slot (110) and each of the first lid slot (122).

13. A cassette according to anyone of claims 9 or 10, wherein each of the second body slot (112) and each of the second lid slot (124) are about 50% narrower and between about 200% to about 500% longer than each of the first body slot (110) and each of the first lid slot (122).

14. A cassette according to anyone of claims 7 to 13, wherein the varied dimensions of slots (110) and (122) relative to slots (112) and (124) optimize reagent flow in the body (104).

15. A cassette according to claim 1, wherein the body (104) further comprises:
a first body chamber (112a) having plurality of second body slots (112);
a second body chamber (112b) having plurality of second body slots (112);
a third body chamber (112c) having plurality of second body slots (112); and
a fourth body chamber (112d) having plurality of second body slots (112).

16. A cassette according to claim 15, wherein the second body slots (112) configured in the first body chamber (112a) are in same direction as of the second body slots (112) configured in the third body chamber (112c).

17. A cassette according to claim 15 or claim 16, wherein the second body slots (112) configured in the second body chamber (112b) are in same direction as of the second body slots (112) configured in the fourth body chamber (112d).

18. A cassette according to anyone of the preceding claims, wherein each of the plurality of first body slots are substantially square in shape, and the second body slots are substantially rectangular in shape.

19. A cassette according to anyone of the preceding claims, wherein the walls of each of the plurality of first body slots are substantially conical and the walls of the second body slots are substantially conical.

20. A cassette according to anyone of the preceding claims, wherein the sample is one of an animal, plant, yeast, bacteria, insect, chemical or synthetic molecule.

21. A cassette according to claim 20, wherein the samples is one of a plasma sample, blood sample, sputum sample, stool sample, lavage, synovial fluid, tissue sample, tumour biopsy sample, cell culture, cytology specimen, synthetic polymer, natural polymer or combinations thereof.

22. A method of processing a sample in vitro, the method comprising the steps of:
i. obtaining a sample (102);
j. placing a sample (102) in a cassette (100) according to anyone of claims 1 to 20;
k. adding suitable for processing of sample reagents to the cassette under step b; and
l. processing the sample and reagent containing cassette under suitable conditions.

23. A method according to claim 22, wherein processing the sample of step d. is under a validated procedure.

24. A method according to claim 22, wherein processing of the sample is under a validated procedure for pathology sample processing.

25. A method according to anyone claim 22 or claim 24, wherein the sample is derived from an animal, plant, yeast, bacteria, insect, chemical or synthetic molecule.

26. A method according to anyone of claims claims 22 to 25, wherein the sample is one of a plasma sample, blood sample, sputum sample, stool sample, lavage, synovial fluid, tissue sample, tumour biopsy, cytology specimen, cell culture, molecular testing or combinations thereof.

27. A method according to claim 26, wherein the method further comprises step e. subjecting the incubated sample of step d. to further analysis such as micromanipulation, microtome manipulations, histology, histopathology, immunohistopathology, cytology, flow cytometry, cell sorting, microscopy, molecular testing, chromatography, microarray analysis, nucleotide sequencing, peptide sequencing, next generation sequencing or combinations thereof.

28. A method of diagnosing a disease or condition in vitro, the method comprising the steps of:
a. obtaining a sample (102);
b. placing a sample (102) in a cassette (100) according to anyone of claims 1 to 21;
c. adding suitable for processing of sample reagents to the cassette under step b;
d. processing the sample and reagent containing cassette under suitable conditions;
e. subjecting the incubated sample of step d. to further analysis such as micromanipulation, microtome manipulations, histology, histopathology, immunohistopathology, cytology, flow cytometry, cell sorting, microscopy, molecular testing, chromatography, microarray analysis, nucleotide sequencing, peptide sequencing, next generation sequencing or combinations thereof; and
f. diagnosing a disease or condition.

29. A method according to claim 28, wherein processing the sample of step d. is under a validated procedure.

30. A method according to claim 29, wherein processing of the sample is under a validated procedure for pathology sample processing.
